(19) United States
(12) Patent Application Publication
(10) Pub. No.: US 2020/0197179 A1
(43) Pub. Date: Jun. 25, 2020

(54) 3D PRINTED CONSTRUCTS FOR CORRECTING BONE DEFECTS AND STEM CELL DELIVERY

(71) Applicant: INDIAN INSTITUTE OF TECHNOLOGY, DELHI, New Delhi (IN)

(72) Inventors: Sourabh GHOSH, New Delhi (IN); Pravesh MEHRA, New Delhi (IN)

(21) Appl. No.: 16/608,804

(22) PCT Filed: Apr. 27, 2018
(86) PCT No.: PCT/IN2018/050264

§ 371 Date: Oct. 25, 2019

(30) Foreign Application Priority Data
Apr. 27, 2017 (IN) 201711015026

(51) Int. Cl. A61F 2/28

(52) U.S. Cl. CPC A61F 2/2803; A61K 35/28

(57) ABSTRACT

A support tray for bone reconstructions has a number of 3D printed constructs of a hydroxyapatite material. Each of the 3D printed constructs are coupled to each other. Each of the 3D printed constructs also has at least one compartment for accommodating regenerating cells at a bone defect site.

### BACKGROUND

The reconstruction of bone defects presents major medical challenges. Such bone defects may arise as a result of either injury, or as a consequence of ablative or reconstructive surgery. Resection of tumorigenic growth on bone portions may also cause removal of certain portions of the bone. In such situations, the defects in the bone may be corrected through techniques referred to as bone reconstruction. Such techniques may generally involve providing metallic or ceramic based spacers or other constructs which may be used for treating such bone defects.

For example, patients suffering from mandibular defect as a result of ameloblastoma, may require resection of a tumour followed by reconstruction of a large part of the mandible. The treatment intends to restore the functional as well as the aesthetic aspects of the face and jaw. Conventionally known approaches, including conservative management to radical resection with or without reconstruction, show 60-80% of recurrence, which in many circumstances may not completely treat facial deformity and function loss of the jaw. In one such example, use of only titanium reconstruction plate with free cortico-cancellous bone grafts can be partly successful in terms of aesthetics and function. However, survival of the bone grafts in the reconstruction plate is a concern. Other major problems associated with the existing methods for mandibular reconstruction are donor site pain, increased tissue morbidity, infection, extra blood loss, aesthetic and functional compromise, higher costs and prolonged surgery time. Thus, the challenge in the management of large ameloblastoma of the mandible is not only to excise tumours to make the patient completely tumor free and to prevent recurrence, but also to provide the best reconstruction, aesthetically and functionally, to drastically reduce cost and surgery time.

### BRIEF DESCRIPTION OF DRAWINGS

The detailed description is described with reference to the following figures. It should be noted that the description and figures are merely examples of the present subject matter and are not meant to represent the subject matter itself.

FIG. 1 depicts a representative image of a 3D printed hydroxyapatite block to be used to fabricate custom-made 3D constructs for forming a support tray, as per an example of the present subject matter;

FIG. 2 depicts a support tray constructed by using 3D printed constructs (FIG. 1) attached by bone cement, as per an example of the present subject matter.

FIG. 3 shows the X-ray image used for the generation of the 3D profile of the tumor site in which the patient-specific construct is to be implanted, as per an example of the present subject matter; Inset image is showing the resected tumor.

FIG. 4 depicts an implanted hydroxyapatite tray post-surgery, as per an example of the present subject matter; and

FIG. 5 illustrates a block diagram of 3D printer used for the fabrication of the 3D printed construct, as per an example of the present subject matter.

### DETAILED DESCRIPTION

The bone is a highly dynamic structural tissue with a complex architecture. A constant supply of growth factors and cytokines are required to stimulate and sustain the growth of this tissue. In the event of minor bone defects, the process of regeneration undergoes normally, and bone development takes its normal course. However, under certain special conditions, such as bone cancer or in the case of large defects caused due to bone trauma, the process of growth and regeneration is inefficient or may not occur at all. In such cases, specialised surgical procedures are needed to initiate the process of regrowth (cell proliferation, differentiation, mineralization) and then sustain the development of bone tissue.

Among the common techniques adopted for surgical treatments of bone defects include bone grafting or the use of metallic plates. In the case of metallic plates, these are only useful upto a point wherein they provide structural support. Such metallic plates, however, only partially support the reconstruction of the defect. In some cases, bone grafting may be accompanied by donor site morbidity.

Bone tissue primarily comprises tissue-specific cell types embedded in a tissue matrix. The primary tissue-specific cells found in the bone are osteocytes, osteoclasts, and osteoblasts. The matrix within which these cells are found comprises a mineral component called hydroxyapatite. Hydroxyapatite forms up to 50% by volume and 70% by weight of human bone and is a derivative of calcium apatite. Besides forming the primary bone structure, the hydroxyapatite rich matrix also contains collagens, glycoproteins, proteoglycans and sialoproteins that provide the organic matrix for the dividing and differentiating bone cells. Commonly used techniques utilizing acrylate-based bone cements, calcium-phosphate based bone cements, solid blocks of hydroxyapatite have certain disadvantages owing to non-porous structure, low resorption rates and lack of migration of essential cells required for re-development of bone tissue.

To this end, various support trays or boxes based on 3D printed constructs are described to achieve bone regeneration in defect-specific manner. In one example, the 3D printed constructs are generated using hydroxyapatite or hydroxyapatite based materials. As per the present subject matter, a plurality of such 3D printed constructs may be assembled together to build a support tray. In one example, the support tray may be built by assembling the 3D printed constructs using bone cement or by suturing together. The number of such 3D printed constructs which may be assembled together to build the support tray may be based on the clinical images of the site of defect or the deformity that is to be treated. Continuing with the present subject matter, each of the 3D printed constructs may further include perforations of a defined dimension, such that when the 3D printed constructs are assembled, they provide for placement of regenerating cells to enable osseointegration and bone formation. The cells of the construct of the present disclosure can be selected from the group consisting of bone marrow-derived mesenchymal stem cells, adipose tissue stem cells and combinations thereof.

In one example, the 3D printed hydroxyapatite construct of the present disclosure is fabricated by using extrusion-based printing methods, such as direct-write assembly of a hydroxyapatite-based bioink. The bioink of the said 3D printed hydroxyapatite construct of the present disclosure is further selected from the group consisting of hydroxyapatite-carboxymethyl cellulose-acrylate, hydroxyapatite-collagen, hydroxyapatite-gelatin, hydroxyapatite-silk and combinations thereof.

The 3D printed hydroxyapatite construct of the present disclosure is fabricated based on a plurality of features including but not limited to the particle size of the hydroxyapatite bioink, the concentration of the hydroxyapatite bioink, the required porosity of the target site of the bone-defect and the size of the bone defect. The hydroxyapatite bioink used for the construct described in the present disclosure has a particle size in the range of 100-900 nanometers and a concentration in the range of 30-60% (volume fraction of the printable bioink). The porosity of the 3D printed hydroxyapatite is optionally in the range of 50-900 micrometer and is modifiable depending on the porosity of bone of the target site. Rheology of the bioink can be optimized in order to print filaments in layer-by-layer manner, so that the filaments are robust enough to span the gaps between two filaments in underlying layers.

As would be understood, the regenerating cells, for example, cells from autologous bone marrow, may develop into bone with concurrent resorption of the support tray (i.e., 3D hydroxyapatite tray) in a sustained manner. The use of regenerating cells and hydroxyapatite enables osseointegration of the surrounding host tissue, and affects new bone formation. Furthermore, over time the 3D hydroxyapatite tray will get degraded in the defect region, and the native bone tissue would be regenerated. The developed hydroxyapatite tray is entirely biocompatible material based, which upon hydrolytic degradation ensures the release of non-immunogenic by-products and thereby, not provoking adverse tissue responses.

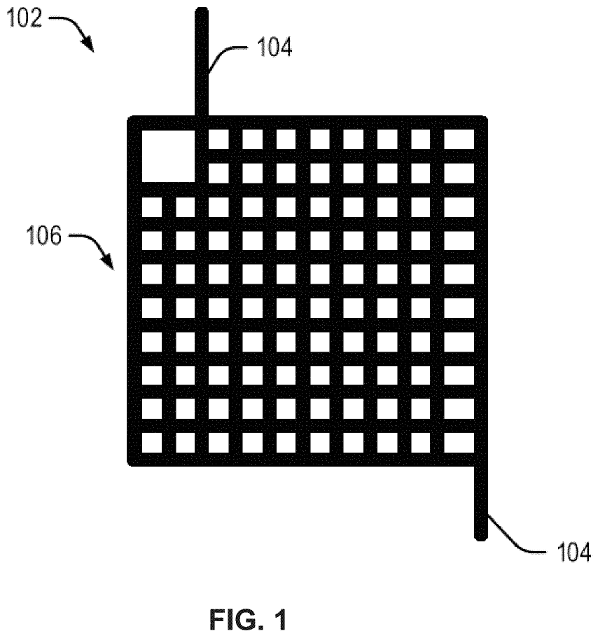

The above-mentioned implementations are further described herein with reference to the accompanying FIGS. 1-5. It should be noted that the description and figures relate to exemplary implementations, and should not be construed as a limitation to the present subject matter. It is also to be understood that various arrangements may be devised that, although not explicitly described or shown herein, embody the principles of the present subject matter. Moreover, all statements herein reciting principles, aspects, and embodiments of the present subject matter, as well as specific examples, are intended to encompass equivalents thereof. It should be further noted that the various examples and implementations are described with respect to bone defects occurring in the mandible. Other bone defects in other parts of the body may also be addressable by the approaches mentioned herein, without any limitation to the present subject matter.

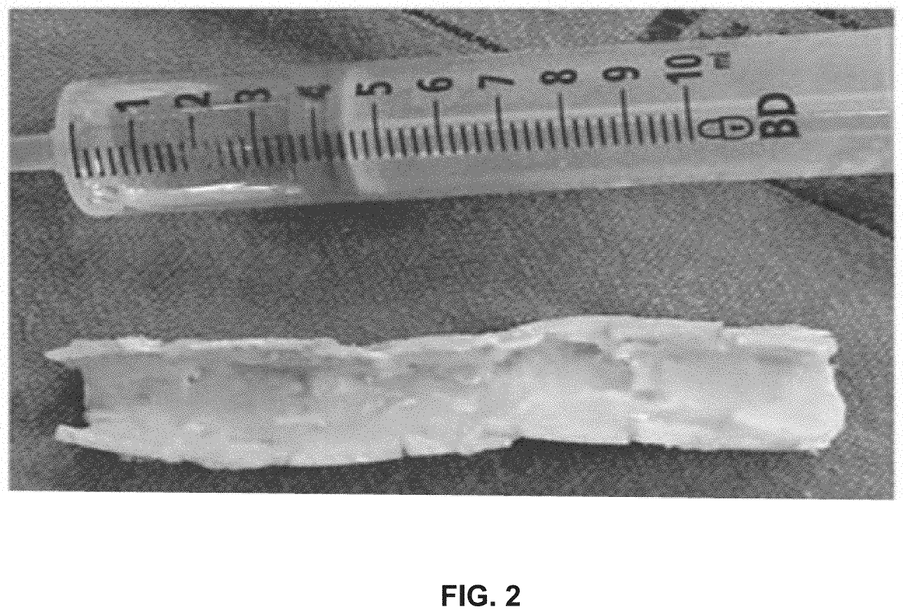

FIGS. 1 and 2 describe one or more 3D printed constructs 102 (referred to as constructs 102) and a support tray 202, respectively. The constructs 102 may be such that multiple constructs 102 may be assembled together to form a support tray 202. In the present example, the construct 102 is constructed using hydroxyapatite and may be fabricated in any size. In another example, the dimensions and porosity of the 3D printed hydroxyapatite constructs can be varied by varying various dimensions associated with the 3D profile and the printing parameters, like printer nozzle diameter, printing rate and extrusion pressure. By regulating the printing parameters both precision and flexibility in fabrication of the 3D printed hydroxyapatite construct can be achieved.

In the example as illustrated, the constructs 102 may be planar in shape with a thickness which is thin as compared to the linear dimensions of the constructs 102. The constructs 102 may further include at least two linear extensions 104 extending from opposing edges of the constructs 102. The extensions 104 may be either of equal length or may be of different lengths, without deviating from the scope of the present subject matter. The extensions 104 provide means for engaging and retaining the constructs 102 with each other, when they are assembled to form support tray 202. In another example, the constructs 102 may be fused together to form the support tray 202.

The constructs 102 may further include a plurality of perforations 106. As would be explained in later sections, the constructs 102 when assembled result in the formation of one or more compartments or spacing owing to the perforations 106. The compartment (not shown in FIG. 2) enable delivery of regenerating cells which in turn aid osseointegration and bone formation.

In another implementation, the design of the constructs 102 may depend on the bone site where the constructs 102 are to be fitted. The support tray 202, interchangeably referred to as the hydroxyapatite tray 202, is constructed using a hydroxyapatite construct scaffold and suitable biodegradable materials. The various constructs 102 may be assembled together to form the hydroxyapatite tray 202 (as shown in FIG. 2). In one example, the hydroxyapatite tray 202 may have provisions for delivering one or more types of regenerating cells for enabling osseointegration of the bone issues and for affecting bone formation. As mentioned previously, the shape of the hydroxyapatite tray 202 may depend on the nature of the bone correction which is to be carried out. For example, in cases where a small portion of bone has been removed, say from the mandible, the size of the hydroxyapatite tray 202 may be small. In cases where the portion of bone removed is more, a corresponding larger sized hydroxyapatite tray 202 would be required.

Assessment of whether a large or a small sized hydroxyapatite tray 202 would be required, may be based on conventional techniques. For example, the dimensions of the defect on the mandible may be obtained using computed tomography imaging (CT). In another example implementation, the size of bone defect may also obtained by X-ray imaging, magnetic resonance imaging, computed tomography and combinations thereof. Once the images of the defect are obtained a 3D profile using computer aided design (CAD) may be generated. This allows the architecture and the dimensions of the construct to be adapted to the defect-specific dimensions, while also conforming to the facial defects of the patient, thus, catering to the aesthetic requirements of the patient in need.

Returning to the structure of the hydroxyapatite tray 202, the hydroxyapatite tray 202 may include one or more compartments for accommodating regenerating cells.

The compartments in turn may be formed as a result of the perforations or holes present in the constructs 102. The constructs 102 when assembled together may result in the formation of such compartments. Further, in accordance with the subject matter, at least one compartment is optionally included in the hydroxyapatite construct for the delivery of progenitor cells at the site of the bone defect. In one example, the cells in the present subject matter may include bone marrow derived mesenchymal stem cells obtained from the iliac crest of a patient. In another example, the regenerating cells (osteoblasts) of the jaw construct of the present disclosure may be selected as well as bone marrow-derived stem cells, adipose tissue stem cells and combinations thereof.

The hydroxyapatite tray 202 may further include one or more projections, i.e., extensions 104 which enable the coupling of the hydroxyapatite tray 202 with the bone tissue. The shape of the hydroxyapatite tray 202 may depend on the bone defect on which the hydroxyapatite tray 202 is being applied. For example, the hydroxyapatite tray 202 when formed for a mandible may have a profile which matches a portion of the jaw bone. The profiling of the hydroxyapatite tray 202 is to ensure appropriate shaping to the facial structure when the hydroxyapatite tray 202 is fitted. As would be noted, the constructs 102, owing to their predefined dimension may be custom-made to form a patient-specific hydroxyapatite tray 202 for mandibular reconstruction. Furthermore, the perforations 106 on the constructs 102 deliver autologous regenerating cells at the defect site. The regenerating cells aid in the development of bone and may further facilitate in concurrent resorption of the hydroxyapatite constructs, such as constructs 102, in a sustained manner, thereby, releasing non-immunogenic by-products and not provoking adverse tissue responses. It has also been observed that the fabrication process for 3D hydroxyapatite tray, such as the hydroxyapatite tray 202, is less costly and surgery involves less time for implanting at the area of bone defect and thereby causing minimal tissue morbidity during implantation of custom made constructs 102.

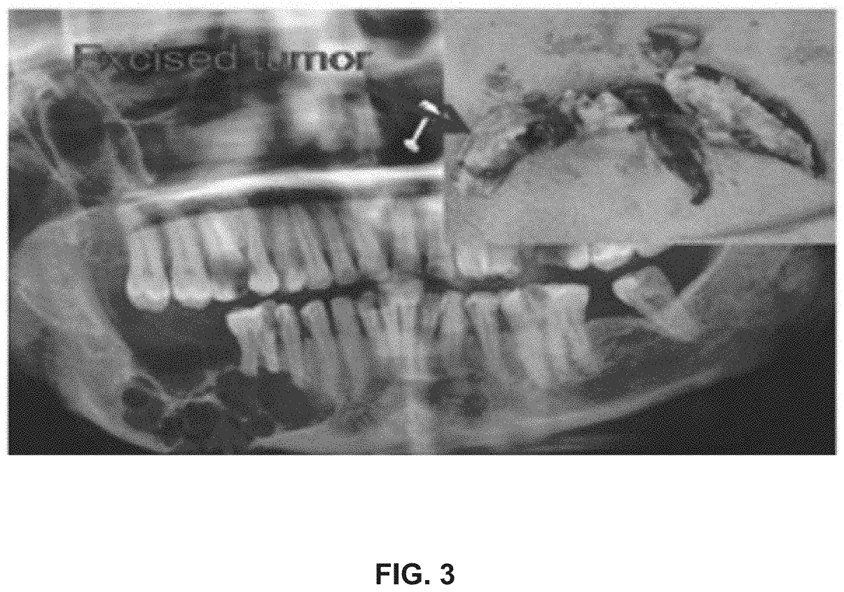

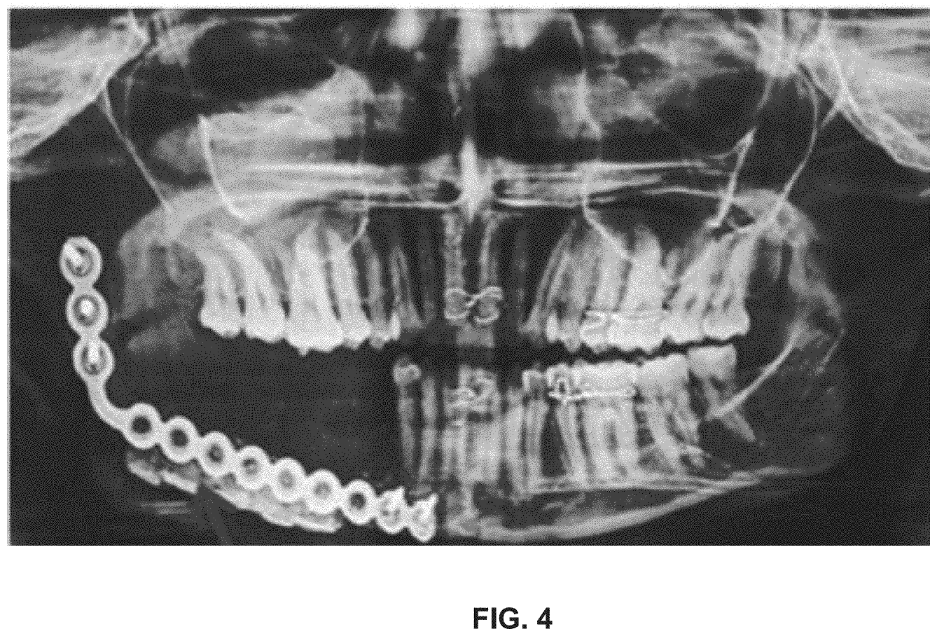

FIGS. 3 and 4 provide X-ray images of mandible with defect which is to be treated using hydroxyapatite tray 202 made from constructs 102. FIG. 3 represents a bone defect in which a substantial portion of the lower mandible has been removed. FIG. 4 depicts the X-ray image of the same portion with the implanted hydroxyapatite tray 202. In the present example, a metallic reconstruction plate may also be used for supporting the hydroxyapatite tray 202 for treating the bone defect. The present approaches involve less surgical time with minimal tissue morbidity and satisfactory post-operative wound healing, thus enabling the patient to carry out daily activities like chewing, talking and swallowing. The hydroxyapatite tray has been found to show no signs of immune rejection with satisfactory clinical form, function, wound healing and aesthetics of patients during the post-operative period. The approaches may be used for large mandibular defect as a result of trauma, recurrent tumor, birth defect or inflammation resulting into better aesthetics and function.

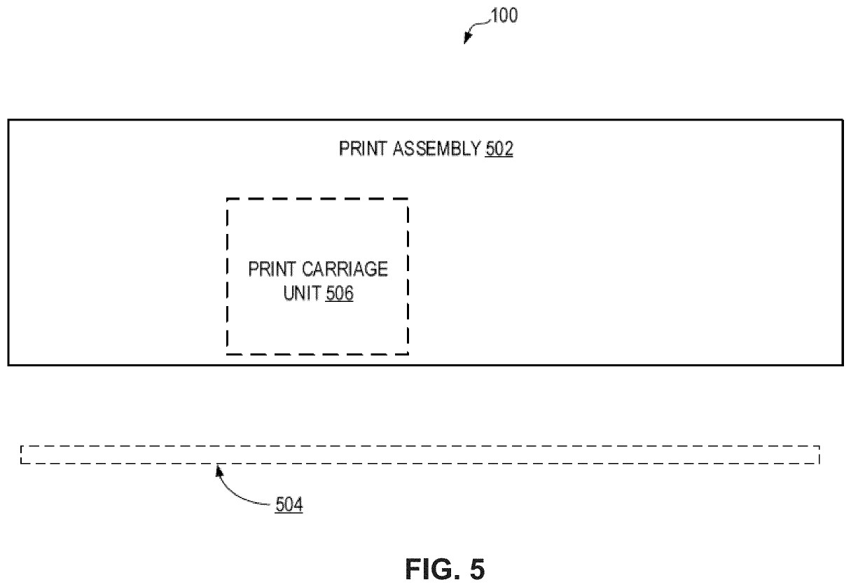

The manner in which the constructs 102 are printed as 3D objects is described in conjunction with FIG. 5. Generally, 3D printing systems are referred to as additive manufacturing systems. FIG. 5 illustrates a block diagram of an additive manufacturing system 500 (referred to as the system 500) for generating one or more 3D objects, such as constructs 502. The block diagram illustrates logical blocks representing functional entities which may be present in the system 500. The block diagram does not indicate any specific arrangement of such functional elements nor does it represent the manner in which such elements may be interconnected with each other. Any arrangement of blocks may be implemented without deviating from the scope of the present subject matter. In the present example, the system 500 includes a print assembly 502 and a work area 504. The print assembly 502 in turn may include a print carriage unit 506. The print assembly 502, i.e., print carriage unit 506, operate over the work area 504 deposit build material and any other suitable agents, layer-by-layer, in order to generate a 3D object, such as constructs 102.

The system 500 performs the 3D printing based on a plurality of printing parameters indicative of printer settings of the 3D printer. The printing parameters include a printer nozzle diameter for the 3D printing and an extrusion pressure at the printer nozzle for the 3D printing. The printer nozzle diameter may range from about 5 micrometers (μm) to about 500 micrometers (μm). The extrusion pressure is the pressure at the printer nozzle at which the bio-ink is extruded during the 3D printing. The extrusion pressure may vary from a range of about 5 pounds per square inch (psi) to 20 psi. The bio-ink of the system of the present subject matter is selected from the group consisting of hydroxyapatite-carboxymethyl cellulose-acrylate, hydroxyapatite-collagen, hydroxyapatite-gelatin, hydroxyapatite-silk and combinations thereof. In one example, the hydroxyapatite bio-ink of the jaw construct of the present subject matter has a particle size in the range of 100-900 nanometers.

The additive manufacturing, i.e., 3D printing, of the constructs 102 is based on a CAD model of the bone defect area where the defect is present. The CAD model may be generated by a computing device coupled to the system 500 and may be based on CT imaging, X-ray imaging, MRI imaging, and combinations thereof.

As mentioned before, the present subject matter is related to a fabricated 3D hydroxyapatite tray, such as the hydroxyapatite tray 202, for supporting and precisely delivering osteoblasts or bone marrow cells that can be implanted in vivo at the area of mandibular defect after surgical resection of the recurrent tumor. The said system can be implanted at the area of defect in less surgical time with minimal tissue morbidity and satisfactory post-operative wound healing, thus enabling the patient to carry out the daily activities like chewing, talking and swallowing. The hydroxyapatite tray shows no signs of immune rejection with satisfactory clinical form, function, wound healing and aesthetics of patients during the post-operative period. The system can be used for large mandibular defect as a result of trauma, recurrent tumor, birth defect or inflammation resulting into better aesthetics and function.

As explained, the present subject matter provides time-saving and cost-saving benefits. The 3D printed tray could be fabricated before surgery. Immediately after removal of tumor, the tray could be fitted in the defect site thereby drastically reducing time of surgery.

Although examples for the present disclosure have been described in language specific to structural features and/or methods, it should be understood that the appended claims are not necessarily limited to the specific features or methods described. Rather, the specific features and methods are disclosed and explained as examples of the present disclosure.

1. A support tray for bone reconstructions, the support tray comprising:
a plurality of 3D printed constructs of a hydroxyapatite material, wherein each of the plurality of 3D printed constructs are coupled to each other and wherein each of the 3D printed constructs further comprises at least one compartment for accommodating regenerating cells at a bone defect site.

2. The support tray for bone reconstructions as claimed in claim 1, wherein said hydroxyapatite material has a concentration in the range of 30% % by volume.

3. The support tray for bone reconstructions as claimed in claim 1, wherein the particle size of the hydroxyapatite material is in the range of about 100 nanometers.

4. The support tray for bone reconstructions as claimed in claim 1, wherein each of the 3D printed constructs has a porosity and pattern design defined based on the bone defect site.

5. The support tray for bone reconstructions as claimed in claim 1, wherein the regenerating cells is one of stem cells, bone marrow stem cells, and adipose-tissue derived stem cells.

6. The support tray for bone reconstructions as claimed in claim 1, having a profile corresponding to the site of the bone defect.

7. The support tray for bone reconstructions as claimed in claim 6, wherein the site of bone defect lies in a mandibular region.

8. The support tray for bone reconstructions as claimed in claim 1, wherein the 3D printed constructs are printed using a hydroxyapatite based bioink.
